(19) United States
(12) Patent Application Publication
(10) Pub. No.: US 2005/0124919 A1
(43) Pub. Date: Jun. 9, 2005

(54) Measuring device

(76) Inventors: James D. Castillo, Los Alamos, CA (US); Bernhard Leitner, Trabuco Canyon, CA (US); Isaac Clinton Reese JR., Trabuco Canyon, CA (US)

Correspondence Address: Kit M. Stetina;STETINA BRUNDA GARRED & BRUCKER, Suite 250, 75 Enterprise, Aliso Viejo, CA, 92656, US

(21) Appl. No.: 10/726,958

(22) Filed: Dec. 3, 2003

(51) Int. Cl. A61B 1/00; G01B 1/00; A61B 5/103; A61B 5/117

(52) U.S. Cl. 600/587; 600/595; 33/512

(57) ABSTRACT

A measuring device for measuring a body part of a patient is described. One embodiment of the measuring device includes a reference component capable of being secured to a body part of a patient. The reference component provides a reference point. An articulated measurement arm is movably coupled to the reference component. The articulated measurement arm has a plurality of sections, each section having a measurement point. A plurality of sensors is associated with the measurement points. The sensors are capable of providing a plurality of data sufficient to allow determination of a position of each measurement point relative to the reference point.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

The present invention relates to a measuring device for measuring a body part of a patient, more particularly a measuring device which can be used to create a computer model of the body part useful in the manufacture of a custom-made brace or the diagnosis of a bodily condition.

Surgery, injury or disease to joints and other parts of the body may require the support provided by a brace or similar device. Braces of varying designs may be used to support a patient's knee, elbow, shoulder, ankle, lower back, neck or other part of the body.

Off-the-shelf products are often the quickest and most cost-effective to obtain. Off-the-shelf braces may be available in different sizes and typically are adjustable to obtain an acceptable fit.

Custom-made braces manufactured to the individual measurements of a particular patient may offer a better fit than off-the-shelf products. A plaster or fiberglass mold of the joint or body part is sometimes used in building a custom-made brace. However, creating a mold is often a lengthy and messy procedure. In addition, the manufacturer of the custom-made brace must have physical access to the mold.

Measurements of the joint or body part are sometimes used in building a custom-made brace. However, most body parts are not regular in shape, thus compounding the problem of making repeatable or meaningful measurements. These measurements also often lack context, or information as to how the measurements relate to each other or a physical landmark.

As described below, the present invention is directed to a measuring device which can provide data sufficient to create a three-dimensional model of a body part without the need for making a mold or making several disparate measurements. These and other objects and advantages of the invention will be described below in connection with the appended drawings illustrating the preferred embodiments of the invention.

### BRIEF SUMMARY OF THE INVENTION

The present invention is directed towards a measuring device, in one embodiment comprising: a reference component capable of being secured to a body part of a patient and providing a reference point; an articulated measurement arm movably coupled to the reference component, the articulated measurement arm having a plurality of sections, each section having a measurement point; and a plurality of sensors associated with the measurement points, the sensors capable of providing a plurality of data sufficient to allow determination of a position of each measurement point relative to the reference point.

In another embodiment measuring device comprises: a femoral component capable of being secured to a leg of a patient and providing a reference point; a tibial component movably coupled to the femoral component, the tibial component capable of being secured to the leg, the tibial component having a first sensor capable of providing data sufficient to determine a position and an orientation of the tibial component with respect to the femoral component; a first measurement arm movably coupled to the femoral component, the measurement arm having a first measurement point capable of being brought into contact with the leg, the first measurement arm having a second sensor capable of providing a plurality of data sufficient to determine a position of the first measurement point with respect to the femoral component; and a second measurement arm movably coupled to the tibial component, the second measurement arm having a second measurement point capable of being brought into contact with the leg, the second measurement arm having a third sensor capable of providing a plurality of data sufficient to determine a position of the second measurement point with respect to the tibial component; wherein the first measurement point and the second measurement point are capable of being in contact with the leg simultaneously.

A method for generating a three-dimensional model of a body part, comprising: establishing a reference point associated with a physical landmark of the body part; bringing a plurality of measurement points into contact with the body part, wherein each of the measurement points is in contact with the body part simultaneously, wherein each of the measurement points is mechanically coupled to the reference point; using a computer to collect data from a plurality of sensors associated with the measurement points, wherein the plurality of sensors are capable of providing a plurality of data sufficient to determine the positions of each measurement point with respect to the reference point; determining the position of each measurement point in three-dimensional space with respect to the reference point to generate the three-dimensional model of the body part.

### BRIEF DESCRIPTION OF THE DRAWINGS

FIG. 1 shows an isometric view of one embodiment of the measuring device of the present invention.

FIGS. 2A-2D show four different views of the measuring device.

FIGS. 3A-3B shows the measuring device with its measurement arms configured to measure a thinner leg and a thicker leg, respectively.

FIG. 4 shows the measuring device in use around the leg of a patient.

### DETAILED DESCRIPTION OF THE INVENTION

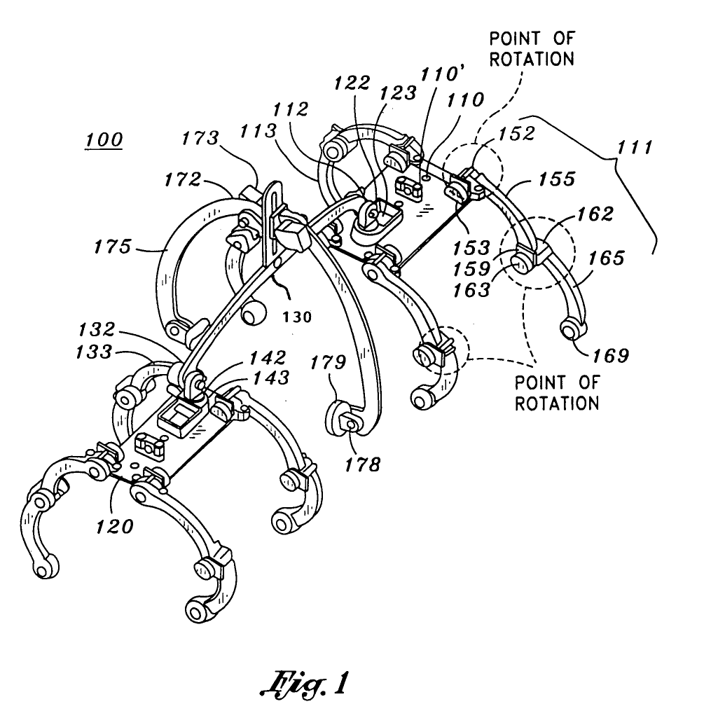

FIG. 1 shows one embodiment of a measuring device 100 of the present invention. Measuring device 100 includes a femoral component 110 and a tibial component 120, both movably coupled to a femoral-tibial component 130. Femoral component 110 includes a reference point 110′.

Femoral component 110 and tibial component 120 each have a surface which allows stable placement on the leg of the patient. Femoral-tibial component 130 is curved to clear the knee of the patient. Femoral component 110, tibial component 120 and femoral-tibial component 130 may be manufactured in different shapes and sizes.

Femoral component 110 and tibial component 120 may include straps or other devices for securely attaching femoral component 110 and tibial component 120 to the leg of the patient. Femoral component 110 and tibial component 120 may also be secured to the leg of the patient using tape, bandages or other non-permanent methods.

A femoral elevation joint 112 and a femoral azimuth joint 122 couple femoral-tibial component 130 to femoral component 110. A tibial elevation joint 132 and a tibial azimuth joint 142 couple femoral-tibial component 130 to tibial component 120. Femoral elevation joint 112 and femoral azimuth joint 122 have axes of rotation which are substantially perpendicular. Tibial elevation joint 132 and tibial azimuth joint 142 also have axes of rotation which are substantially perpendicular. Femoral elevation joint 112, femoral azimuth joint 122, tibial elevation joint 132 and tibial azimuth joint 142 may allow the force required to move femoral component 110 and tibial component 120 to be fixed or adjustable. Femoral elevation joint 112, femoral azimuth joint 122, tibial elevation joint 132 and tibial azimuth joint 142 are hinges, but may be universal joints, ball-and-socket joints, or any other suitable coupling devices.

A femoral elevation sensor 113 and a femoral azimuth sensor 123 are coupled to femoral elevation joint 112 and femoral azimuth joint 122, respectively. Femoral elevation sensor 113 and femoral azimuth sensor 123 are capable of detecting an elevation angle and an azimuth angle, respectively, of femoral-tibial component 130 with respect to femoral component 110. A tibial elevation sensor 133 and a tibial azimuth sensor 143 are coupled to tibial elevation joint 132 and tibial azimuth joint 142, respectively. Tibial elevation sensor 133 and tibial azimuth sensor 143 are capable of detecting an elevation angle and an azimuth angle, respectively, of femoral-tibial component 130 with respect to tibial component 120. Femoral elevation sensor 113, femoral azimuth sensor 123, tibial elevation sensor 133 and tibial azimuth sensor 143 are angular potentiometers, but may also be any suitable sensor or group of sensors capable of providing sufficient data to allow the positions and orientations femoral component 110, tibial component 120 and femoral-tibial component 130 to be determined with respect to reference point 110′.

Femoral component 110, tibial component 120 and femoral-tibial component 130 are rigid and have known geometries. Data from femoral elevation sensor 113, femoral azimuth sensor 123, tibial elevation sensor 133 and tibial azimuth sensor 143 are used with these known geometries to calculate the positions and orientations of femoral component 110, tibial component 120 and femoral-tibial component 130 with respect to reference point 110′.

Measurement arms 111 are movably coupled to femoral component 110 and tibial component 120. Measurement arms 111 are articulated, each having an inner section 155 and an outer section 165. Inner section 155 and outer section 165 are curved to accommodate a wide range of patients, but may also be made in any suitable shape or size. Four measurement arms 111 are coupled to each of femoral component 110 and tibial component 120, but any number of measurement arms 111 may be coupled to femoral component 110 and tibial component 120. Measurement arms 111 are coupled to femoral component 110 and tibial component 120 at substantially right angles, but may also be coupled at any angle or each at different angles. Each measurement arm 111 may also have a single section or articulated into any number of sections. Each measurement arm 111 may also be of a telescoping design.

An inner section joint 152 movably couples inner section 155 to femoral component 110 or tibial component 120. An outer section joint 162 movably couples outer section 165 to inner section 155. Inner section joint 152 and outer section joint 162 may allow the force required to move inner section 155 and outer section 165 to be fixed or adjustable. Inner section joint 152 and outer section joint 162 are hinges which limit the motion of inner section 155 and outer section 165 to one axis, but may also be universal joints, ball-and-socket joints or any other suitable coupling devices.

An inner section sensor 153 and an outer section sensor 163 are coupled to inner section joint 152 and outer section joint 162, respectively. Inner section sensor 153 is capable of detecting an angle between inner section 155 and femoral component 110 or tibial component 120. Outer section sensor 163 is capable of detecting an angle between outer section 165 and inner section 155. Inner section sensor 153 and outer section sensor 163 are angular potentiometers, but may also be any suitable sensor or group of sensors capable of providing sufficient data to allow the positions and orientations of inner section 155 and outer section 165 with respect to femoral component 110 or tibial component 120 to be determined.

An inner section measurement point 159 and an outer section measurement point 169 are located at the ends of inner section 155 and outer section 165, respectively. Each inner section measurement point 159 and each outer section measurement point 169 are capable of being in contact with the leg of the patient simultaneously with all other measurement points. Inner section measurement point 159 and outer section measurement point 169 may also be attached anywhere along inner section 155 and outer section 165 respectively. Inner section 155 and outer section 165 may also have multiple measurement points.

Inner section 155 and outer section 165 are rigid and have known geometries. Data from inner section sensor 153 and outer section sensor 163 are used with these known geometries to calculate the positions of inner section measurement point 159 and outer section measurement point 169 with respect to femoral component 110 or tibial component 120.

Knee measurement arms 175 are movably coupled to femoral-tibial component 130. Knee measurement arms 175 are curved to accommodate a wide range of patients, but may also be made in any suitable shape or size. Two knee measurement arms 175 are coupled to femoral-tibial component 130, but any number of knee measurement arms 175 may be coupled to femoral-tibial component 130. Knee measurement arms 175 are coupled to femoral-tibial component 130 at substantially right angles, but may also be coupled at any angle or each at different angles. Knee measurement arms 175 may also be articulated into two or more sections. Knee measurement arms 175 may also be of a telescoping design.

A knee measurement arm joint 172 movably couples each knee measurement arm 175 to femoral-tibial component 130. Knee measurement arm joint 172 may allow the force required to move knee measurement arm 175 to be fixed or adjustable. Knee measurement joint 172 is a hinge which limits the motion of knee measurement arm 175 to one axis, but may also be a universal joint, ball-and-socket joint or any other suitable coupling device.

A knee measurement arm sensor 173 is coupled to knee measurement arm joint 172. Knee measurement arm sensor 173 is capable of detecting an angle between knee measurement arm 175 and femoral-tibial component 130. Knee measurement arm sensor 173 is an angular potentiometer, but may also be any suitable sensor or group of sensors capable of providing sufficient data to allow the position and orientation of knee measurement arm 175 to be determined with respect to femoral-tibial component 130.

A knee measurement point 179 is located at the end of each knee measurement arm 175. Each knee measurement point 179 is capable of being in contact with the leg of the patient simultaneously with all other measurement points. Knee measurement point 179 is a cup designed to locate a condyle of the patient, but may also be any other suitable shape. Knee measurement point 179 is movably coupled to knee measurement arm 175 with a swivel 178, but may also be fixed. Knee measurement point 179 may also be located anywhere along knee measurement arm 175. Knee measurement arm 175 may also have multiple measurement points.

Knee measurement arm 175 is rigid and has a known geometry. Data from knee measurement arm sensor 173 is used with this known geometry to calculate the position and orientation of knee measurement point 179 with respect to femoral-tibial component 130.

Measuring device 100 may be calibrated by first using it on a cylinder of known diameter. Measuring device 100 may then be secured to the leg of the patient and adjusted so that all measurement points are in contact with the leg of the patient at the same time. Data from all sensors is obtained substantially simultaneously by a computer connected to the sensors.

The positions of all inner section measurement points 159 and outer section measurement points 169 with respect to femoral component 110 and tibial component 120 are known. The positions of all knee measurement points 179 with respect to femoral-tibial component 130 are known. The positions and orientations of femoral component 110, tibial component 120 and femoral-tibial component 130 with respect to reference point 110′ are known. As a result, the positions of all inner section measurement points 159, outer section measurement points 169 and knee measurement points 179 with respect to reference point 110′ may be calculated.

The reference point may be used as an origin in a three-dimensional space, and the positions of each of the measurement points with respect to the reference point, as well as the positions and orientations of femoral component 110 and tibial component 120, used as coordinate data to generate a three-dimensional computer model of the leg of the patient. This model may be transmitted to the brace manufacturer to aid in the manufacture a custom-made knee brace. This model may also be used to diagnose varus and valgus knees.

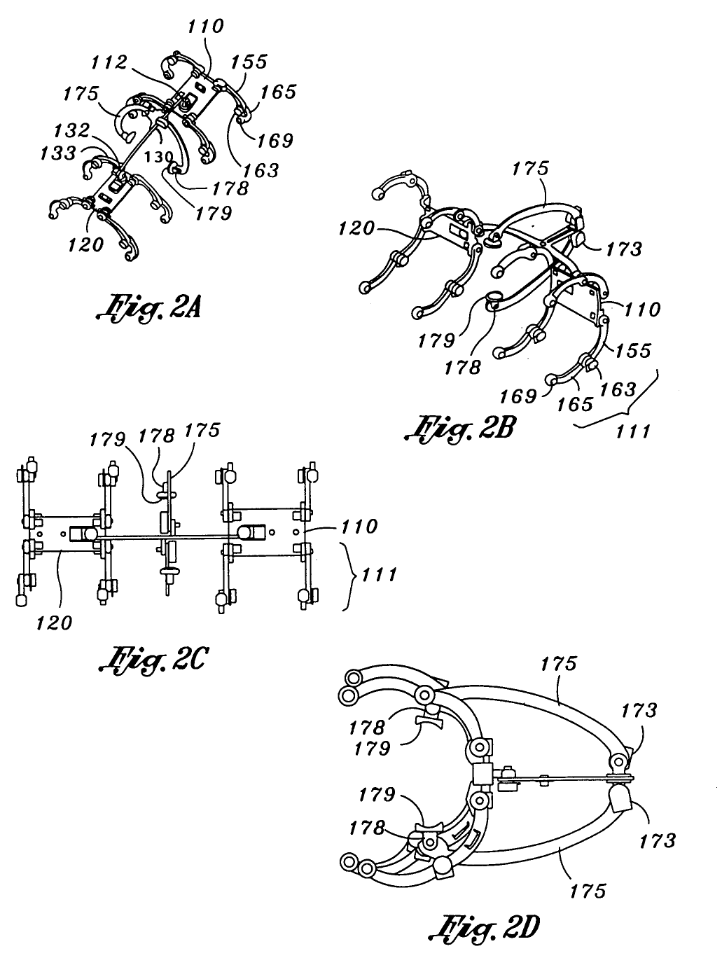

FIGS. 2A-2D show four different views of measuring device 100. FIG. 2A is an isometric view from above. FIG. 2B is an isometric view from below. FIG. 2C is a view from directly above. FIG. 2D is a view from head-on.

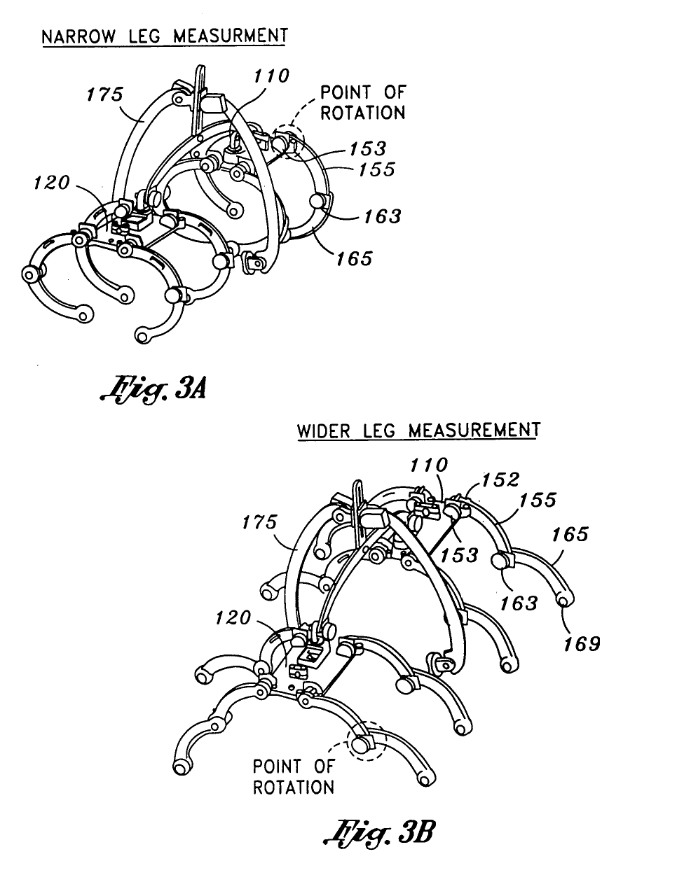

FIGS. 3A-3B shows measuring device 100 with its measurement arms 111 adjusted as if to measure a thinner leg and a thicker leg, respectively.

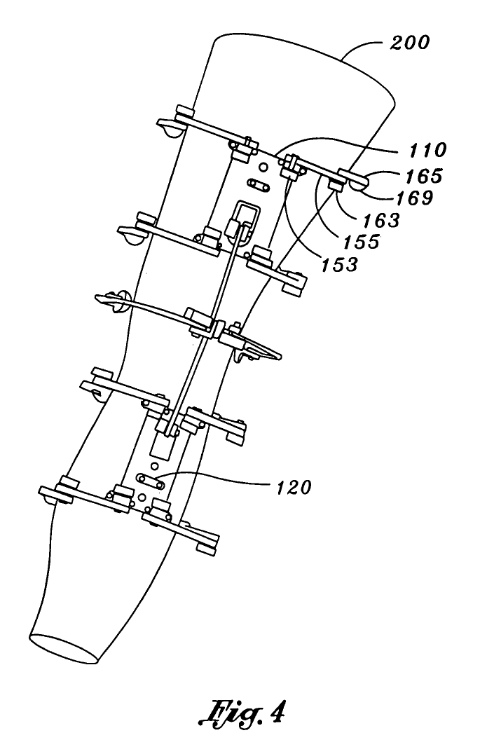

FIG. 4 shows measuring device 100 in use around a leg 200 of a patient. Typically, measuring device 100 is used with the knee in a straight and locked. However, measuring device 100 may also be used to make measurements of a bent knee.

The basic design of measuring device 100 may be used for measuring devices for other parts of the body. For example, a measuring device for the neck may be simplified, having only a reference component and one or more articulated measurement arms movably coupled to the reference component, and suitable sensors for detecting the positions of the measurement arms with respect to the reference component.

Although for purposes of illustration, certain materials, components, and structural embodiments have been depicted, those skilled in the art will recognize that various modifications to the same can be made without departing from the spirit of the present invention, and such modifications are clearly contemplated herein.

1. A measuring device, comprising:
a reference component capable of being secured to a body part of a patient and providing a reference point;
an articulated measurement arm movably coupled to the reference component, the articulated measurement arm having a plurality of sections, each section having a measurement point; and
a plurality of sensors associated with the measurement points, the sensors capable of providing a plurality of data sufficient to allow determination of a position of each measurement point relative to the reference point.

2. The measuring device of claim 1, wherein the articulated measurement arm is movably coupled to the reference component with a hinge.

3. The measuring device of claim 1, wherein at least one of the sensors is an angular potentiometer.

4. The measuring device of claim 1, wherein the measurement points are capable of being in contact with the body part simultaneously.

5. The measuring device of claim 1, wherein the plurality of data is provided and captured by a computer substantially simultaneously.

6. A measuring device, comprising:
a femoral component capable of being secured to a leg of a patient and providing a reference point;
a tibial component movably coupled to the femoral component, the tibial component capable of being secured to the leg, the tibial component having a first sensor capable of providing data sufficient to determine a position and an orientation of the tibial component with respect to the femoral component;
a first measurement arm movably coupled to the femoral component, the measurement arm having a first measurement point capable of being brought into contact with the leg, the first measurement arm having a second sensor capable of providing a plurality of data sufficient to determine a position of the first measurement point with respect to the femoral component; and
a second measurement arm movably coupled to the tibial component, the second measurement arm having a second measurement point capable of being brought into contact with the leg, the second measurement arm having a third sensor capable of providing a plurality of data sufficient to determine a position of the second measurement point with respect to the tibial component;
wherein the first measurement point and the second measurement point are capable of being in contact with the leg simultaneously.

7. The measuring device of claim 6, wherein the first measurement arm is articulated and includes a first inner section movably coupled to the femoral component and a first outer section movably coupled to the first inner section, wherein the first measurement point is coupled to the first inner section, the measuring device further comprising:
a third measurement point coupled to the first outer section; and
a fourth sensor capable of providing a plurality of data sufficient to determine a position of the third measurement point with respect to the first measurement point;
wherein the first measurement point and the third measurement point are capable of being in contact with the leg simultaneously.

8. The measuring device of claim 6, wherein the second measurement arm is articulated and includes a second inner section movably coupled to the tibial component and a second outer section movably coupled to the second inner section, wherein the second measurement point is coupled to the second inner section, the measuring device further comprising:
a fourth measurement point coupled to the second outer section; and
a fifth sensor capable of providing a plurality of data sufficient to determine a position of the fourth measurement point with respect to the second measurement point;
wherein the second measurement point and the fourth measurement point are capable of being in contact with the leg simultaneously.

9. The measuring device of claim 6, wherein the first sensor is a plurality of sensors.

10. The measuring device of claim 6, wherein the first sensor is at least one angular potentiometer.

11. The measuring device of claim 6, wherein the tibial component is coupled to the femoral component with a hinge.

12. The measuring device of claim 6, wherein the first measurement arm is coupled to the femoral component with a hinge.

13. The measuring device of claim 6, wherein the second measurement arm is coupled to the tibial component with a hinge.

14. The measuring device of claim 6, further comprising:
a femoral-tibial component, wherein the femoral component and the tibial component are movably coupled to femoral-tibial component, the femoral tibial component having a sixth sensor capable of providing a plurality of data sufficient to determine a position and an orientation of the femoral-tibial component with respect to the femoral component.

15. The measuring device of claim 6, further comprising:
a third measurement arm movably coupled to the femoral-tibial component, the third measurement arm having a fifth measurement point capable of being brought into contact with the leg, the third measurement arm having a seventh sensor capable of providing a plurality of data sufficient to determine a position of the fifth measurement point with respect to the femoral-tibial component;
wherein the first measurement point, second measurement point and fifth measurement point are capable of being in contact with the leg simultaneously.

16. A method for generating a three-dimensional model of a body part, comprising:
establishing a reference point associated with a physical landmark of the body part;
bringing a plurality of measurement points into contact with the body part, wherein each of the measurement points is in contact with the body part simultaneously, wherein each of the measurement points is mechanically coupled to the reference point;
using a computer to collect data from a plurality of sensors associated with the measurement points, wherein the plurality of sensors are capable of providing a plurality of data sufficient to determine the positions of each measurement point with respect to the reference point; and
determining the position of each measurement point in three-dimensional space with respect to the reference point to generate the three-dimensional model of the body part.
